(19) United States
(12) Patent Application Publication
(10) Pub. No.: US 2003/0086821 A1
(43) Pub. Date: May 8, 2003

(54) Apparatus for the treatment and destruction of harmful pathogens enclosed in postal and delivery items

(76) Inventor: Robert R. Matthews, Richmond, CA (US)

Correspondence Address: T. Gene Dillahunty, BURNS, DOANE, SWECKER & MATHIS, L.L.P., P.O. Box 1404, Alexandria, VA, 22313-1404, US

(21) Appl. No.: 10/027,852

(22) Filed: Dec. 21, 2001

Related U.S. Application Data
(60) Provisional application No. 60/337,564, filed on Nov. 8, 2001.

(51) Int. Cl. A61L 2/10; A61L 2/20

(52) U.S. Cl. 422/29; 422/28; 422/186.07; 422/186.08; 422/24; 422/292; 422/300; 422/305

(57) ABSTRACT

The present invention provides systems and methods for destroying pathogens (including bacteria and viruses) from delivery, postal or courier items such as letters, parcels, boxes, etc. In preferred aspects, the items to be treated are placed in a chamber which is then flooded with ozone gas at pressures above ambient atmospheric pressure. The exposure to pressurized ozone is carried out at a sufficiently high concentration, and for a sufficiently long duration such that the sterilizing ozone penetrates into the interior of the delivery, postal or courier item.

### RELATED APPLICATIONS

The present application claims the benefit of U.S. Provisional Patent Application No. 60/337,564, filed Nov. 8, 2001 and U.S. Provisional Patent Application No., (Unassigned), Attorney Docket No. 033472-002, filed Oct. 26, 2001.

### FIELD OF THE INVENTION

The present invention relates to the handling of mail, courier or other delivery services in general, and to systems for ensuring the safety of such mail, courier or other delivery services from biological contamination in particular.

### BACKGROUND OF THE INVENTION

In the handling and delivery of the mail, undesirable and lethal pathogens (e.g.: anthrax) can intentionally or accidentally be coated on the outside of the envelope or packaging material or placed on the inside of the envelope or packaging. Such biogens can be life threatening to people who come in contact with the pathogens through either inhalation, ingestion, or cutaneous exposure (i.e. penetration of the skin). Unfortunately, there is no equipment currently used to kill these pathogens and render them harmless. Moreover, since the handlers of the mail can not see, smell, touch, or taste these pathogens due to their microscopic size, there are no human senses which can detect their presence. Exposure to these pathogens can lead to illness and or death if not treated early with the proper antibiotics. Consequently, systems are needed to sterilize mail, envelopes, parcels and packages for safe handling.

One approach to treating the pathogens is to expose the letter or package to gamma radiation from radioactive isoptopes. Typically Cesium 137 or Cobalt 60 are the isotopes used. There are numerous disadvantages of such systems. For example, the isotopes must be carefully used because the device handling these isotopes itself emits radiation. In addition, because the power source of the device is radioactive, there are environmental impacts with the radioactive isotope source disposal and recycling.

Other sterilization approaches use electrical energy in the form of either Electron Beams or X-Rays. Such approaches, including gamma radiation, electron beams, and X-Rays all irradiate the pathogens and cause death by breaking the chemical bonds of the pathogens DNA chain. Unfortunately, such sterilization equipment is expensive, it requires skilled operators, and it can be hazardous to system operators. Moreover, shielding must be used to contain the irradiation energy which would otherwise be hazardous to operators who came in contact with the energy. A disadvantage of such systems is that even accidental exposure can lead to cancer forming entities within humans. Moreover, such equipment is typically limited in its ability to handle large or irregularly shaped objects.

Still other sterilization approaches use chlorine (i.e. bleach). Chlorine kills microorganisms by oxidation. The active form of chlorine in water is hypochlorous acid, HOCl. The mechanism of bacteria destruction by chlorine requires diffusion of chlorine through the cell wall of the bacterium. The oxidation of the bacterium's enzymes by the OCl⁻ ion then brings about death. Unfortunately, the diffusion of chlorine in the form of HOCL through the cell wall, and subsequent death of the bacterium, is a process that occurs in the time frame of 30-60 minutes. Moreover, a further disadvantage is that such a liquid sterilization system would not be suitable for paper products, such as letters or parcels.

Alternatively, the use of ozone in water achieves sterilization via a direct attack on the microorganism's cellular membrane and does not involve hydrolysis. Rather, ozone ruptures the cell wall (lysing) and the cytoplasm is lost into the environment. Advantageously, the organism can not develop an immunity to the lysing process. Also, the rate of bacteria kill by ozone has been shown by Hulluta to be 3,125 times faster than by chlorine (Hulluta, Das Gas und Wasserfach, vol 44, p. 1261 (1963)). Unfortunately, a disadvantage of ozonated water sterilization is that it cannot be used to sterilize paper products including envelopes, letters or packages.

### SUMMARY OF THE INVENTION

The present invention encompasses a variety of systems which can be used to sterilize mail (e.g. letters and envelopes) or parcels or packages or boxes or other paper products (e.g. currency or personal or traveler's checks) or bubblewrap pouches or any other delivery item through exposure to pressurized ozone gas. The present invention can also be used to destroy pathogens on other delivery items, such as flower, gift or foodstuff deliveries. It is to understood, therefore, that the present references to “mail” or “parcels” or “packages” are merely exemplary, and that the present invention is not so limited.

In accordance with the present invention, the mail, parcel or package is first placed in a chamber. Pressurized ozone is then added to the chamber. The ozone remains in the chamber under pressure for a period of time sufficient to penetrate into the mail, parcel or package and sterilize the contents therein. In particular, the ozone gas used in the present invention sterilizes pathogens by oxidizing the pathogens.

The Applicant has surprisingly discovered that pressurized ozone gas diffuses quickly and effectively through the wrappings of standard envelopes, parcels or packages and effectively kills biological pathogens therein.

Accordingly, the present invention preferably comprises raising the pressure of the gas solution which enables a sufficiently high ozone concentration in gas phase to provide oxidation of substantially all of the pathogens residing inside and outside the envelope, parcel or package. Thus, the present invention eliminates the need to use hazardous chemicals or energies in the destruction of pathogens from mail.

In an exemplary aspect of the present invention, the Applicant has determined that a concentration of ozone of 150 g/m3 (which is about 150,000 ppm ozone), at a pressure of ozone of about 1795 Torr, when maintained for about 3 minutes is sufficient to penetrate and sterilize the contents of a standard letter envelope. In various preferred aspects, however, the ozone concentration may range from 50,000 ppm to 350,000 ppm ozone.

Of course, the ability of pressurized ozone to penetrate into the interior of various envelopes or packages will depend upon the material used. Thus, in accordance with the present invention, different packaging materials require different sterilization routines. Thus, the present invention comprises sterilization routines which vary as to: (1) the pressure of the ozone in the sterilization chamber, (2) the concentration of ozone in the atmosphere in the chamber, (3) the duration of time over which the exposure to pressurized ozone is maintained, and (4) the particular material through which the ozone need penetrate.

Thus, it is to be understood that the pressures and times of exposure described herein are merely exemplary and that higher pressures, concentrations and exposure times are needed to penetrate thicker/heavier packaging materials (such as cardboard or shrinkwrap). Additionally, sterilization can be achieved at higher ozone pressures, which require reduced exposure times, and vice versa.

Moreover, various pathogens have different forms which require different durations and concentrations of the ozone exposure for their destruction. For example, the anthrax pathogen may either be in the form of a bacterium or a spore. In the case of a anthrax bacterium, a relatively short duration, low concentration of ozone may be all that is required to destroy the anthrax. Conversely, in the case of an anthrax spore, a much longer duration, higher concentration of ozone is required to destroy the anthrax. Thus, the duration and concentration of the ozone to which the letter or parcel is exposed depends upon the form of the pathogen therein.

Thus, although various applications of the preferred method employ an ozone exposure of less than 5 minutes, it is to be understood that exposures as long as 30 minutes, or longer, are all encompassed within the scope of the present invention. Moreover, the desired ozone exposure time will also increase depending upon the expected amount of pathogen destroyed. For example, a greater exposure time will be used to destroy 99.999% of the pathogen than would be used to destroy 99.9% of the pathogen.

Additionally, the greater the difference in ozone concentration between the exterior and the interior of the letter or parcel, the faster the ozone will migrate into the letter or parcel, thereby sterilizing the pathogens therein. Thus, by exposing the letter or parcel to much higher ozone concentrations, the duration of such exposure can be markedly reduced. Therefore, increased volumes of mail can be sterilized in the same period of time.

Although the present invention is not so limited, the Applicant's present invention preferably comprises the following preferred ozone pressures, concentrations and exposure times, (as discovered by the Applicant's own testing), for each of the following packaging materials:

(1) Letters in standard envelopes containing one to twenty sheets of paper therein:

an ozone pressure of about 1795 Torr, at a concentration of about 150 g/m3, (about 150,000 ppm) for a duration of about 2 to 5 minutes, and most preferably about 3 minutes.

(2) Cardboard Packaging:

an ozone pressure of about 1795 Torr, at a concentration of about 150 g/m3, (about 150,000 ppm) for a duration of about 5 minutes.

(3) Plastic Shrinkwrap:

an ozone pressure of about 1795 Torr, at a concentration of about 150 g/m3, (about 150,000 ppm) for a duration of about 3 minutes.

(4) Unwrapped Dollar Bills:

an ozone pressure of about 1795 Torr, at a concentration of about 150 g/m3, (about 150,000 ppm) for a duration of about 1 minute.

(5) Unwrapped Computer Disks:

an ozone pressure of about 1795 Torr, at a concentration of about 150 g/m3, (about 150,000 ppm) for a duration of about 1 minute.

As stated above, different concentrations of ozone gas may be needed to sterilize different forms of pathogens. For example, a short duration, low concentration of ozone may destroy an anthrax bacterium, whereas a high duration, high concentration of ozone will be needed to destroy an anthrax spore.

In various preferred aspects of the present invention, an ozone concentration as low as 3 ppm may be used. However, a preferred ozone concentration between 50,000 ppm and 350,000 ppm may also be used. In one optional aspect, a preferred ozone concentration of about 150,000 ppm is used.

It is to be understood that the above exposure durations and concentrations (and the pressures required to achieve such concentrations) are exemplary. Suitable ozone durations and concentrations for sterilization will, of course, depend upon the depth of packaging through which the ozone must pass to reach the pathogen. Thus, if the letters or parcels are simply sterilized when stacked or piled one upon another, the ozone must penetrate through several letters or parcels to reach the bottom of the stack or pile. In such a case, greater ozone exposure durations and concentrations will be required. As will be more fully explained, an advantage of the present invention is that it separates individual letters or parcels such that ozone flow occurs around the individual letters or parcels, thus reducing the ozone exposure duration and concentration required to achieve sterilization.

In accordance with the present invention, the ozone introduced into the chamber in which the mail/packaging is placed is at a pressure greater than ambient such that the ozone penetrates fully through the packaging.

In accordance with the present invention, a variety of systems can be used to generate the ozone. For example, in one preferred embodiment of the invention, a coronal discharge ozone generator is used to generate the ozone which is introduced into the sterilization chamber/chamber. In alternate preferred aspects, ultraviolet (UV) light may be used to generate the ozone or oxygen free radicals (for example, UV at a wavelength of 172 nm or 185 nm or 254 nm). It is to be understood that the present invention encompasses any system which produces or otherwise supplies the ozone. Moreover, pure oxygen or ambient air or any other suitable oxygen supplying source or mixture may be used as an input to either the coronal discharge or UV light ozone generation systems.

Preferably, after the pressurized ozone is removed from the sterilization chamber/chamber, it is destroyed such that it cannot be released to the environment. In one preferred embodiment of the invention, pellets made of copper oxide, manganese dioxide and aluminum oxide are used to destroy the ozone such that it is not inadvertently released to the environment after it has been removed from the sterilization chamber. In another preferred embodiment of the invention, a UV light (for example, UV at a wavelength of 254 nm) is used to destroy the ozone such that it is not released to the environment after it has been removed from the sterilization chamber.

In preferred aspects, the present invention includes a pressurized sterilization chamber in which one or many mail letters or package are placed for sterilization. In optional preferred aspects, individual letters may be positioned on support racks such that they are spaced apart from one another. In alternate aspects, the individual letters/packages are placed within a rotary drum which is then spun such that ozone circulates fully between (and thus penetrates into) the tumbling letters/parcels.

In optional preferred aspects, a transport system may be provided for moving a number of letters/packages into and/or out of the sterilization chamber. Transport of the mail through this chamber can be performed in either a vertical or horizontal mode. In those optional aspects of the present invention in which ultraviolet lamps are used for ozone generation within the sterilization chamber environment, the ultraviolet lamps would be parallel to the direction of mail movement.

In optional preferred aspects, the present invention further comprises systems for introducing moisture into the sterilization chamber. An advantage of introducing such moisture is that it can re-hydrate spores (such as anthrax spores), thereby opening the spore and making ozone penetration more efficient.

In optional preferred aspects, the present invention further comprises systems for Ph control. Such systems may preferably include systems for introducing organic acids or trace amounts of HCL gas. In optional preferred aspects, the compounds which are used to adjust the Ph are bubbled through a water or acid solution and then introduced as a fine moisture into the sterilization chamber. An advantage of controlling the Ph is that it can be adjusted to maintain an environment which is particularly unsuitable for pathogens (such as anthrax spores) to survive.

Accordingly, the present invention advantageously provides a system and apparatus capable of killing pathogens within a mail or delivery handling environment.

Another advantage of the present invention is that it provides a system and apparatus capable of killing pathogens in the mail which does not require the mail envelopes/packages to be opened.

Another advantage of the present invention is that it provides a system which is unaffected by the shape or size of the package(s) placed therein. Instead, an advantage of the present gas sterilization processes is that the gas has the ability to conform to irregular shaped objects without affecting the transfer of ozone to the pathogen inside or on the surface of the package(s). Thus, a further advantage of the present invention is that it separates individual letters or parcels from one another, permitting ozone circulation therearound.

Ozone is an unstable compound which has a short half-life. Thus it does not leave a long term residue on sterilized items.

Another advantage of the present invention is that it provides a system and apparatus which is environmentally safe.

These and other advantages of the present invention will become apparent upon a review of the following specification and the claims appended thereto.

### BRIEF DESCRIPTION OF THE DRAWINGS

FIG. 1 is a schematic illustration of a first embodiment the present invention.

FIG. 2 is an illustration similar to FIG. 1, but showing a cut-away view of a first embodiment of the pressurized sterilization chamber assembly.

FIG. 3A is a front cut-away view of the sterilization chamber of FIG. 2.

FIG. 3B is a side cut-away view of the sterilization chamber of FIG. 2.

FIG. 4 is an illustration similar to FIG. 1, but showing a cut-away view of a second embodiment of the sterilization chamber assembly.

FIG. 5 is a cut-away view of a third embodiment of the sterilization chamber assembly, with conveyors positioned to load and unload the device.

FIG. 6 is a perspective view of a conveyor sterilization system.

FIG. 7 is an exploded view of a preferred embodiment of a mail/parcel tumbler.

FIGS. 8A and 8B are side sectional views of the tumbler of FIG. 7 in an open and closed position, respectively.

FIG. 9 shows a schematic of a system which sterilizes both postal tubs/trays and the letter or parcels stored therein.

FIGS. 10A and 10B are sequential schematic illustrations of a system for separating a plurality of envelopes to permit ozone flow around each envelope without altering the orientation or sequence of the envelopes.

### BEST MODE OF CARRYING OUT THE INVENTION

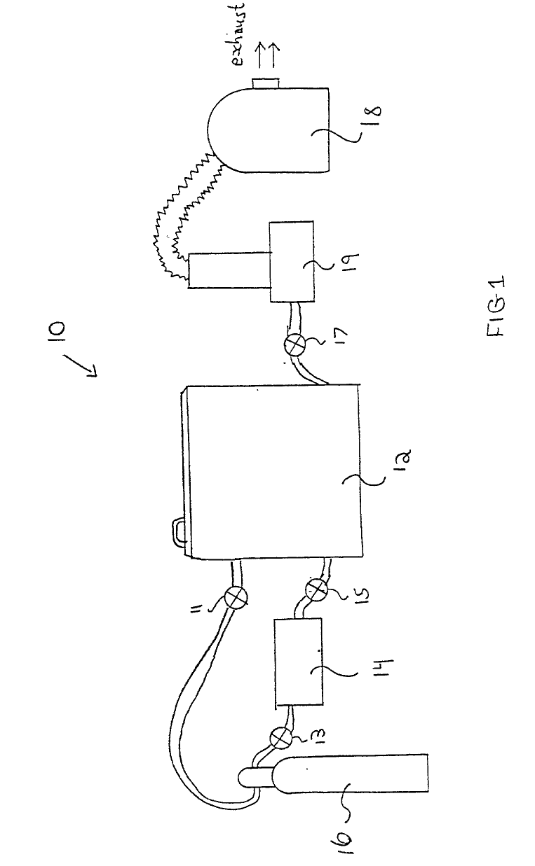

Referring first to FIG. 1, sterilization system 10 is provided. In accordance with the present invention, mail, packages, parcels, etc. are sterilized by exposure to pressurized ozone. Specifically, such mail, packages or parcels are first placed in container/chamber 12. Chamber 12 is then sealed shut. An ozone generator 14 is used to generate ozone. Ozone generator 14 may comprise a coronal discharge generator, but any suitable ozone generator is contemplated. A tank 16 supplies pressurized air or oxygen which is used to maintain ozone at a sufficient pressure within chamber 12.

Valves 13 and 15 are opened (while valve 17 is closed) and ozone generator 14 turned on such that the mail, packages or parcels placed within chamber 12 are exposed to pressurized gasses having a high enough concentration of ozone for a suitable period of time (such that the ozone has penetrated through the envelope of packaging and sterilized its internal contents). After sterilization, valve 15 is closed and valve 17 opened such that chamber 12 is depressurized. Preferably, a suction device or vacuum 18 is then used to empty chamber 12 of ozone. Optionally, a valve 11 can be opened such that the oxygen in tank 16 enters chamber 12 as ozone is removed therefrom. Optionally, the contents of chamber 12 can be opened to the atmosphere as ozone is removed therefrom.

Vacuum 18 extracts the pressurized contents of chamber 12 through ozone destruction device 19. In preferred aspects, ozone destruction device 19 comprises a catalytic destruct unit, for example, a cylinder comprising pellets which are a mixture of copper oxide, manganese dioxide and aluminum oxide. It is to be understood that any suitable ozone destruction system is contemplated within the scope of the present invention.

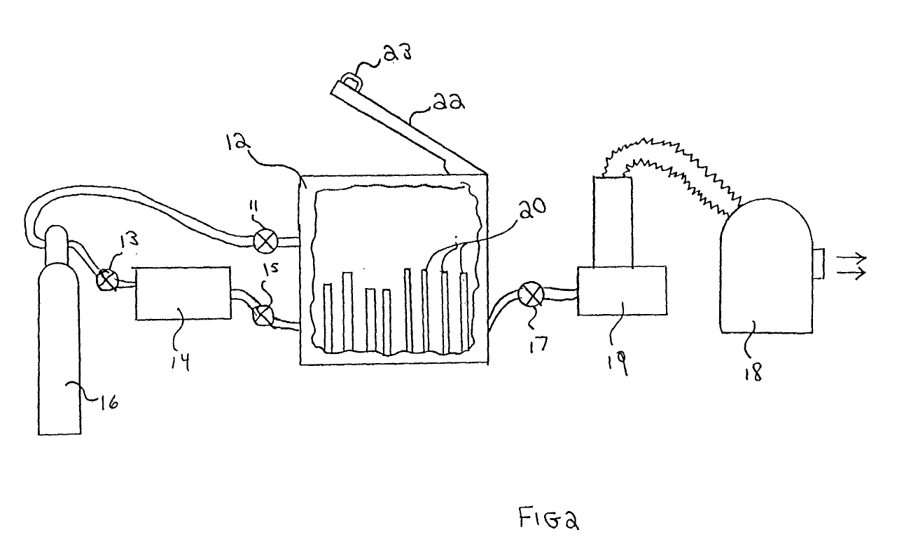

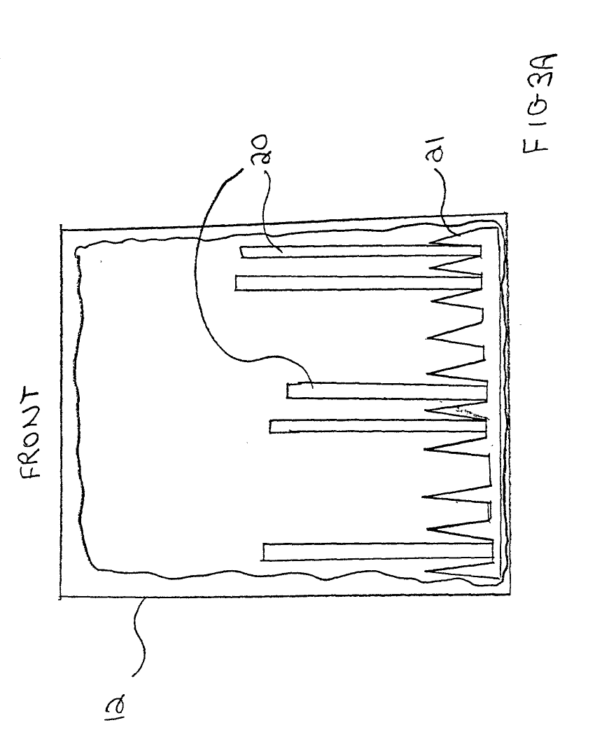

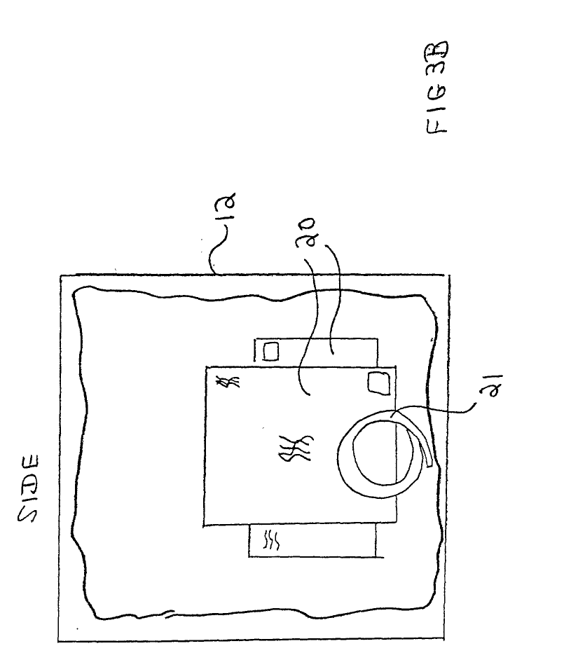

As shown in cut-away view of FIGS. 2, 3A and 3B, chamber 12 may have a plurality of envelopes 20 standing therein. Envelopes 20 may be placed standing side-by-side in rack 21 (which may comprise a spiral coil of wire). An advantage of using a rack 21 is that individual envelopes stand and are thus separated from one another, permitting pressurized ozone gas to circulate therebetween. Access into chamber 12 is provided by opening door 22 with handle 23.

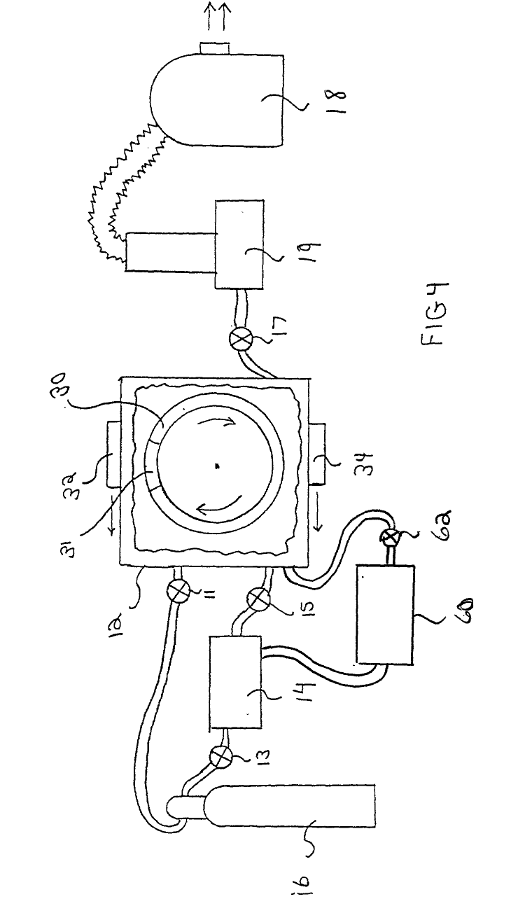

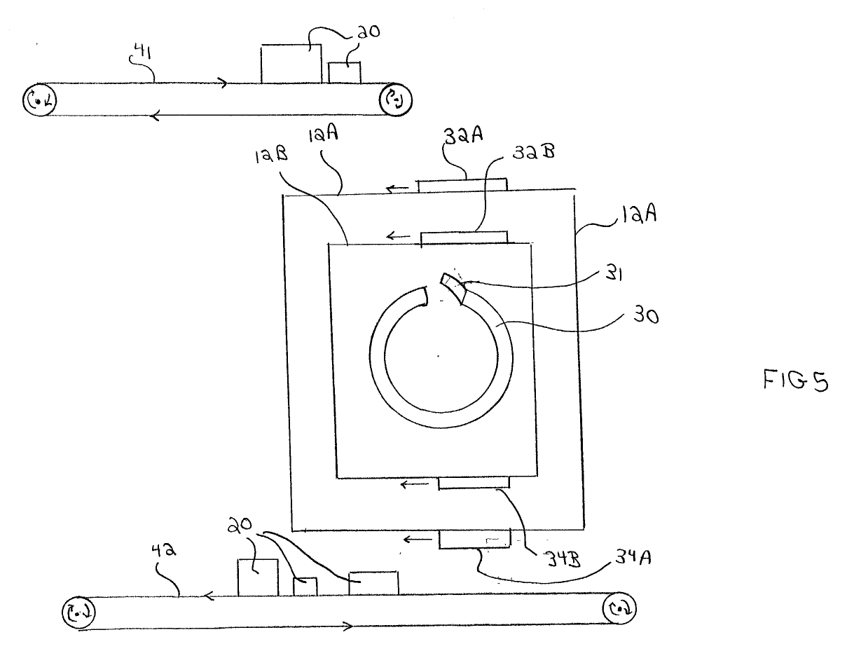

Alternatively, as shown in FIGS. 4 and 5, chamber 12 may comprise a rotary tumbler 30 into which a number (or mixed assortment) of letters, envelopes packages or parcels are placed. Tumbler 30 may preferably simply comprise a tubular wire mesh with a door 31 therein. An advantage of using tumbler 30 is that as individual envelopes are tossed around in the tumbler, the pressurized ozone gas circulated between the letters. A further advantage of rotary tumbler 30 is that since ozone is heavier than air, it tends to settle at the bottom of chamber 12. Thus, as tumbler 30 rotates, the letters or packages tumbling therein will stir up the air in chamber 12, thereby preventing the ozone from merely settling at the bottom of chamber 12.

As is also seen in the second embodiment of the invention in FIG. 4, chamber 12 may have a top door 32 and a bottom drawer 34. Doors 32 and 34 may be sliding doors as shown or they may be hinged doors. Tumbler 30 preferably has an internal door 31, permitting the tumbler to be filled with mail or packages. At the start of operation, door 31 is preferably first positioned under sliding door 32. Both doors 31 and 32 are opened and mail or parcels are loaded into tumbler 30. Chamber 12 is pressurized with ozone. After sterilization has been completed, tumbler 30 is rotated such that door 31 is positioned next to bottom door 34. Both doors 31 and 34 are then opened such that the sterilized contents of tumbler 30 are emptied out of the bottom of chamber 12.

Optionally included in this system is a device 40 which may introduce any of moisture, organic acids or trace amounts of HCL gas into chamber 12. In one aspect, device 40 comprises a water or acid solution through which ozone from ozone generator 14 is bubbled. It is to be understood, however, that device 60 is not so limited, but instead may comprise any system for adjusting or varying the moisture or Ph content within chamber 12. Valve 62 is provided for flow regulation.

FIG. 5 illustrates a dual-wall sterilization chamber for use in accordance with the present invention. Specifically, chamber 12A comprises a dual wall structure with an external top door 32A and an external bottom door 34A. Chamber 12A houses an internal chamber 12B having its own top door 32B and a bottom door 34B. Sterilization is performed in chamber 12B while chamber 12A operates as a secondary containment chamber to prevent accidental ozone leaks (from sterilization chamber 12B) into the room.

As seen in FIG. 5, an advantage of having top and bottom doors into chamber 12 is that mail or parcels may conveniently be sterilized in batches. Specifically, an upper conveyor 41 can load letters/parcels 20 into the top of the device when double doors 32A and 32B are opened; and, a lower conveyor 42 can unload letters/parcels 20 from the bottom of the device when double doors 34A and 34B are opened. An advantage of using such a “flow through” tumbler sterilization process is that one side of the process (i.e. exiting conveyor 42) can be kept “clean”, whereas only the other side of the process (i.e. entering conveyor 41) is regarded as “dirty”. This advantage is achieved since sterilized mail is never brought back up through the same path when non-sterilized mail enters the sterilization chamber.

Thus, the present invention ideally suited for use in a night drop off facility or corporate mailroom whereby all mail on the “dirty” side is considered to be hazardous, and an operator need only come into contact with the “clean” side of the system.

In accordance with the present invention, ozone and oxygen free radicals produced from a corona discharge device (or by a UV light) can be used for sterilization. In the destruction of the pathogens, the process of the present invention results in oxidation of organic materials present to gases of CO_{X}, NO_{X}, O₂, and O₃.

In order to obtain sufficiently high ozone concentration inside the envelope or parcel in a short time frame, the ozone concentration preferably is greater than 3 ppm, and more preferably between 50,000 ppm and 350,000 ppm.

In addition, the pressure of the ozone gas is preferably greater than ambient pressure (i.e. 760 Torr at standard temperature). In accordance with preferred aspects of the present invention, however, typical operating pressures may range from 760 Torr to 3,800 Torr.

In accordance with the present invention, a temperature in the range of −35° C. to 60° C. is appropriate, and is thus not necessary to control. Preferably, however, preferred operating temperatures range from 15° C. to 25° C.

In various preferred aspects, the ozone will be diffused into the chamber for about 1 second to about 15 minutes depending on the load and size of the mail to be processed. In one preferred batch type embodiment, the ozone is diffused (ie: entered into the sterilization chamber) for 2 to 5 or about 5 to 10 minutes. It is to be understood, however, that exposure times as long as 30 minutes, or even longer are contemplated within the scope of the present invention. When using a continuous stream of mail flow, the ozone would preferably be diffused continuously.

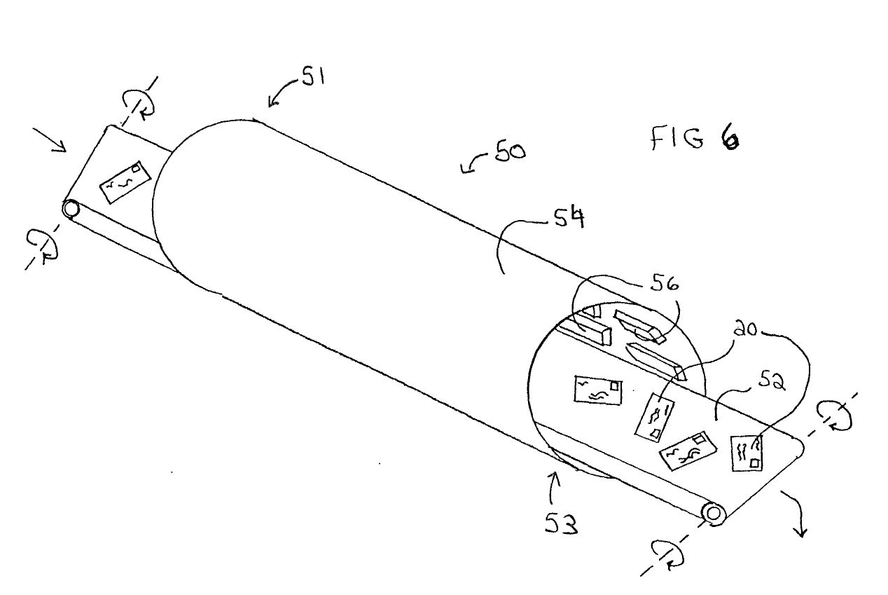

An example of a section of a continuous conveyor sterilization system is shown in FIG. 6. Sterilization system 50 comprises a conveyor belt 52 on which are placed. Conveyor belt 52 moves letters/parcels 20 through tube 54. On the interior of tube 54 are UV lights 56. The UV lights 56 which are positioned at the entrance 51 and/or along through most of the length of tube 54 are operated at a wavelength which produces ozone. This ozone is used to sterilize the contents of letters 20. Conversely, the UV lights 56 which are positioned towards the end 53 of tube 54 are preferably operated at a wavelength of light which destroys ozone. Thus, letters 20 may be passed along through tube 54 at a speed such that they remain exposed to the ozone in tube 54 for a suitable period of time for sterilization to be achieved. Therefore, the length of time letters 20 are exposed to sterilizing ozone will depend both on the length of tube 54 and the speed of conveyor 52.

It is to be understood that tube 54 as shown in FIG. 6 represents a section of length of a suitable sterilization tube. Thus, tube 54 could be much longer than as illustrated. Moreover, tube 54 (and sterilization system 50) may represent a component in a larger sterilization system. For example, systems which seal both ends of tube 54 such that it can be pressurized are also contemplated.

The system illustrated in FIG. 6 may also be operated without UV lights 56. In this aspect of the invention, pressurized ozone is simply supplied to (e.g. injected into) tube 54. In this aspect of the invention, the exhaust from tube 54 may be removed through a catalytic destruction unit (e.g. device 19 in FIG. 1).

An advantage of using UV lights is that the ultraviolet radiation from the ultraviolet light both produces low levels of ozone, and also excites the ozone gas to form the oxygen free radicals which are more reactive than ozone gas molecules. The combined ozone gas and oxygen free radicals react quickly to oxidize the pathogens on or inside the envelope or package.

At the exit end 53 of tube 54, envelopes 20 may optionally be passed through an ultra violet light curtain (arrangement of lights 56) to decompose any unreacted ozone which converts the molecules back to oxygen.

In yet another preferred aspect, the mail passes first by a UV light source while entering the sterilization chamber/tube where by both ozone gas and atomized water are simultaneously injected therein. This mist provides a solvent for ozone absorption. The UV light, ozone gas, and water mist all conform to various shapes and sizes of envelopes or packages passing through the system.

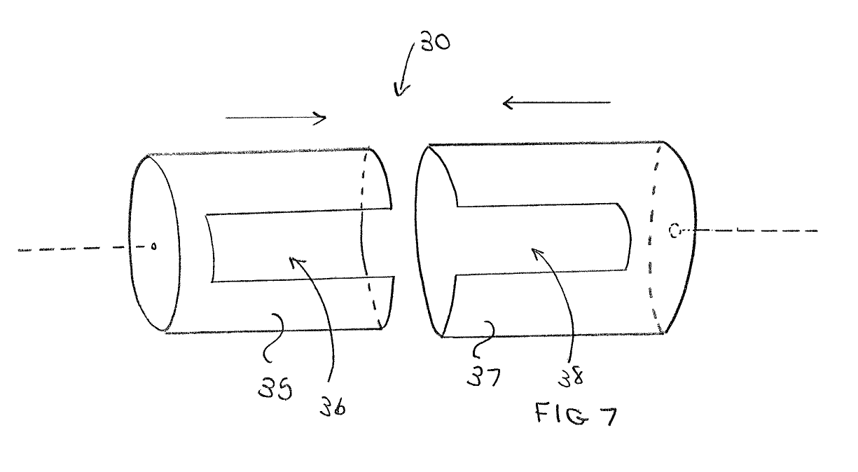

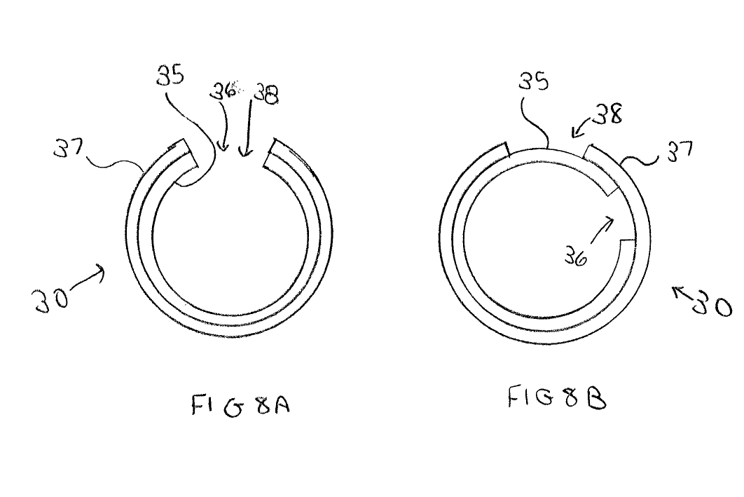

An optional preferred embodiment of mail/parcel tumbler 30 is shown in FIGS. 7 to 8B. FIG. 7 shows an exploded view of tumbler 30 in which a first portion 35 is received into a second portion 37. Portion 35 has a longitudinal slot 36 running therealong and portion 35 has a longitudinal slot 36 running therealong. Portions 35 and 37 are separately rotatable such that when positioned as shown in FIG. 8A, slots 36 and 38 are aligned. When slots 36 and 38 both face upwardly, mail can be loaded into tumbler 30. Conversely, when slots 36 and 38 both face downwardly, mail can be unloaded from tumbler 30. Thus, alignment of slots 36 and 38 operates similar to opening a single door 31 in tumbler 30 (see FIGS. 4 and 5). When portions 35 and 37 are rotated to the position shown in FIG. 8B, tumbler 30 is closed and can then be rotated to tumble its contents in the presence of pressurized ozone therein.

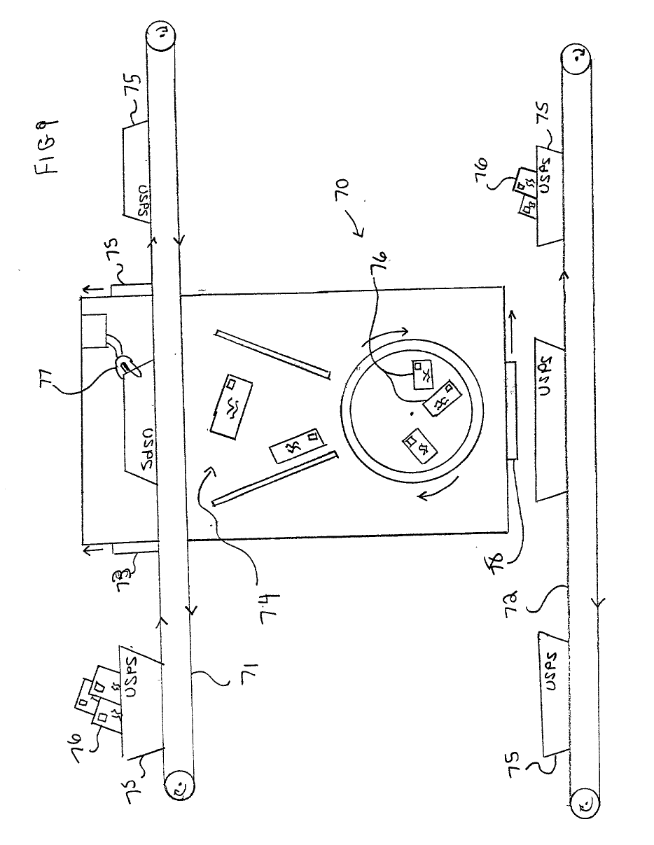

FIG. 9 illustrates a system 70 which sterilizes both letters and parcels and the containers (e.g.: bins or tubs or trays) in which they are transported. This is particularly advantageous since unsterilized equipment (e.g.: standard USPS bins and trays) may still permit cross contamination if only the contents (ie: the mail itself) is sterilized.

When door 73 is opened, in input conveyor 71 transfers a tub (or bin or tray) 75 carrying letters 76 into sterilization chamber 74. Mechanism 77 dumps the contents out of tub 75 and into tumbler 30 for sterilization. It is to be understood that mechanism 77 may comprise any system for emptying the contents of tub 71.

As can be seen, both tub 75 and letters 76 are received within the sterilization chamber 74. Thus, a single sterilization chamber 12 may be used to sterilize both tub 75 and letters 76. In optional aspects, however, separate sterilization chambers may be used for each of letters 76 and tub 75. For example, an upper sterilization chamber may be used to sterilize tub 75 and a lower sterilization chamber may be used to sterilize letters 76.

After sterilization is complete, door 75 is opened and conveyor 71 moves sterilized tub 75 out of chamber 74. Concurrently, output conveyor 72 is used to position a “clean” tub 75 under door 78 so that bin 75 can be loaded to remove sterilized letters 76 from system 70. It is to be understood that although separate conveyors and bins may be used to load “dirty” mail into system 70 and to remove “clean” mail from system 70, the present invention is not so limited. Specifically, the present invention also includes systems in which mail is removed from a tub, the mail and the tub are both sterilized, and the mail is then reloaded into the same bin before being removed from the device.

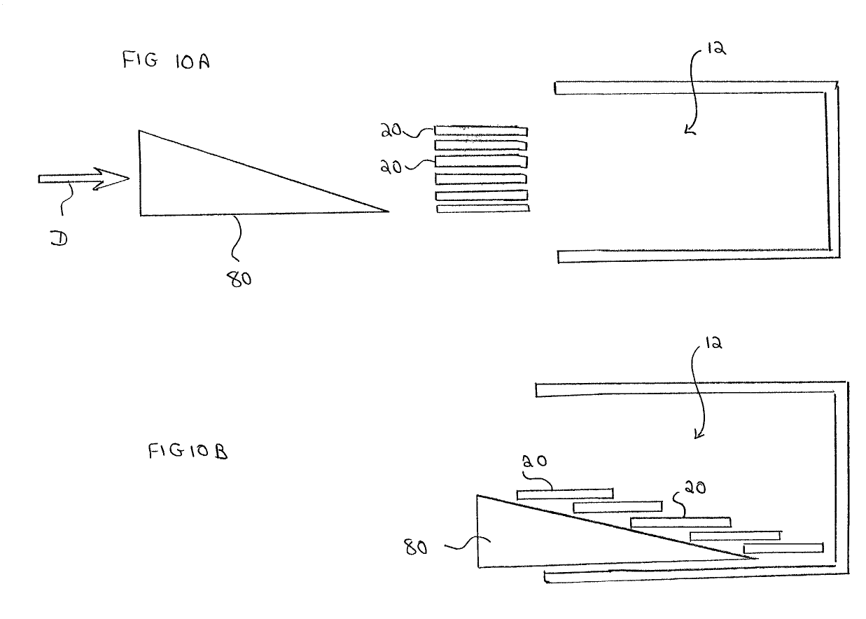

In optional aspects, the present invention also includes systems which separate a plurality of individual envelopes, thereby permitting ozone flow around each envelope without altering the orientation or sequence of the envelopes. Referring to FIG. 10A, a stack of letters 20 is positioned in front of sterilization chamber 12. Thereafter, as shown in FIG. 10B, wedge 80 is moved in direction D such that is positioned letters 20 within chamber 12 while still maintaining the order and orientation of the individual letters. This optional feature of the present invention is particularly advantageous in that it is not necessary to re-sort the letters after sterilization. It is to be understood that the optional aspect of the invention shown in FIGS. 10A and 10B can be incorporated into the various embodiments of the present invention, described herein.

1. A method of sterilizing a delivery item, comprising:
exposing the delivery item to a concentration of ozone such that the ozone penetrates into the delivery item and sterilizes pathogens in an interior of the delivery item.

2. The method of claim 1, wherein exposing the delivery item to a concentration of ozone comprises exposing the delivery item to pressurized ozone.

3. The method of claim 1, wherein the concentration of ozone is at least 3 ppm.

4. The method of claim 1, wherein the concentration of ozone is between 50,000 ppm and 350,000 ppm.

5. The method of claim 4, wherein the concentration of ozone is about 150,000 ppm.

6. The method of claim 1, wherein the duration of exposure is at least 1 minute long.

7. The method of claim 1, wherein the duration of exposure is between 2 and 5 minutes long.

8. The method of claim 1, wherein the duration of exposure is about 3 minutes long.

9. The method of claim 1, further comprising:
generating the ozone by exposing oxygen to ultraviolet light.

10. The method of claim 1, further comprising:
destroying the ozone by exposing the ozone to ultraviolet light.

11. The method of claim 1, wherein the delivery item comprises a postal item.

12. A method of sterilizing letters in standard envelopes, comprising:
exposing the envelopes to an ozone concentration between 50,000 ppm and 350,000 ppm for a duration of about 2 to 5 minutes.

13. The method of claim 12, wherein the concentration of ozone is about 150,000 ppm.

14. The method of claim 12, wherein the duration is about 3 minutes.

15. The method of claim 12, wherein the envelopes are exposed to an ozone pressure of about 1795 Torr.

16. A method of sterilizing paper through cardboard packaging, comprising:
exposing the cardboard packaging to an ozone concentration between 50,000 ppm and 350,000 ppm for a duration of about 5 minutes.

17. The method of claim 16, wherein the concentration of ozone is about 150,000 ppm.

18. The method of claim 16, wherein the cardboard packaging is exposed to an ozone pressure of about 1795 Torr.

19. A method of sterilizing paper through plastic shrinkwrap, comprising:
exposing the plastic shrinkwrap to an ozone concentration between 50,000 ppm and 350,000 ppm for a duration of about 3 minutes.

20. The method of claim 19, wherein the concentration of ozone is about 150,000 ppm.

21. The method of claim 19, wherein the shrinkwrap is exposed to an ozone pressure of about 1795 Torr.

22. A method of sterilizing unwrapped currency, comprising:
exposing the currency to an ozone concentration between 50,000 ppm and 350,000 ppm for a duration of about 1 minute.

23. The method of claim 22, wherein the concentration of ozone is about 150,000 ppm.

24. The method of claim 22, wherein the shrinkwrap is exposed to an ozone pressure of about 1795 Torr.

25. A method of sterilizing unwrapped computer disks, comprising:
exposing the unwrapped computer disks to an ozone concentration between 50,000 ppm and 350,000 ppm for a duration of about 1 minute.

26. The method of claim 25, wherein the concentration of ozone is about 150,000 ppm.

27. The method of claim 25, wherein the shrinkwrap is exposed to an ozone pressure of about 1795 Torr.

28. The method of claim 1, wherein the delivery item is exposed to pressurized ozone when positioned in a first sealed chamber.

29. The method of claim 28, wherein the delivery item is placed into a rotating tumbler within the first sealed chamber.

30. The method of claim 28, wherein the first sealed chamber is positioned within a second sealed chamber.

31. The method of claim 1, wherein the delivery item is exposed to pressurized ozone when moved along through a chamber.

32. The method of claim 31, wherein the delivery item is moved by a conveyor.

33. The method of claim 1, further comprising:
exposing the delivery item to moisture.

34. The method of claim 1, further comprising:
exposing the delivery item to an acidic environment.

35. A method of sterilizing a delivery item, comprising:
exposing the delivery item to a concentration of ozone for a time sufficient for the ozone to sterilize pathogens on the delivery item.

36. An apparatus for sterilizing a delivery item, comprising:
a sterilization chamber dimensioned to receive the delivery item therein; and
a system for introducing a concentration of ozone into the sterilization chamber such that the ozone penetrates into the delivery item and sterilizes pathogens in an interior of the delivery item.

37. The system of claim 36, wherein the system for introducing a concentration of ozone into the sterilization chamber comprises a system for introducing pressurized ozone into the sterilization chamber.

38. The system of claim 36, wherein the system for introducing a concentration of ozone comprises:
a coronal discharge ozone generator which supplies ozone into the sterilization chamber.

39. The system of claim 36, further comprising:
a rotary tumbler within the sterilization chamber.

40. The system of claim 36, wherein the system for introducing a concentration of ozone comprises:
at least one ultraviolet light adapted to generate ozone.

41. The system of claim 36, further comprising:
at least one ultraviolet light adapted to destroy ozone.

42. The system of claim 36, further comprising:
a transport system positioned to load the delivery item into the sterilization chamber.

43. The system of claim 36, further comprising:
a transport system positioned to remove the delivery from the sterilization chamber.

44. The system of claim 36, further comprising:
a transport system positioned to move the delivery item through the chamber.

45. The system of claim 36, further comprising:
an exhaust gas removal system for removing exhaust gas from the chamber.

46. The apparatus of claim 45, wherein the exhaust gas removal system comprises a catalytic system which removes oxidized organic materials from the exhaust gas.

47. The system of claim 39, wherein the rotary tumbler comprises two separately rotatable portions, each portion having a elongated slot passing therealong, and wherein one portion of the tumbler is received into the other portion of the tumbler.

48. The system of claim 36, further comprising:
a system for introducing moisture into the sterilization chamber.

49. The system of claim 36, further comprising:
a system for altering the Ph in the sterilization chamber.

50. The system of claim 36, wherein the sterilization chamber is also dimensioned to receive a container in which a plurality of the delivery items are transported, further comprising:
a system for emptying delivery items from the container, prior to sterilizing both the container and the delivery item.

51. The system of claim 36, further comprising:
a sterilization chamber dimensioned to receive a container in which a plurality of the delivery items are transported; and
a system for emptying delivery items from the container, prior to sterilizing both the container and the delivery item.
